(19) United States
(12) Patent Application Publication
(10) Pub. No.: US 2019/0038853 A1
(43) Pub. Date: Feb. 7, 2019

(54) DEVICE AND A METHOD FOR CONTROLLED ADMINISTERING OF A THERAPEUTIC COMPOSITION TO A PATIENT

(71) Applicant: CANABOLABS, Or-Akiva (IL)

(72) Inventors: Aharon M. EYAL, Jerusalem (IL); Shay Avraham SARID, HodHasharon (IL)

(73) Assignee: CANABOLABS, Or-Akiva (IL)

(21) Appl. No.: 16/155,543

(22) Filed: Oct. 9, 2018

Related U.S. Application Data
(63) Continuation-in-part of application No. PCT/IB2017/052065, filed on Apr. 10, 2017.
(60) Provisional application No. 62/320,541, filed on Apr. 10, 2016.

(51) Int. Cl. A61M 11/04; A61M 15/00

(52) U.S. Cl. CPC A61M 11/042; A61M 15/0003; A61M 2205/3368; A61M 2205/3584; A61M 2205/52; A61M 2205/8206; A61M 2205/3592

(57) ABSTRACT

Controlling device particularly adapted for administering a therapeutic composition, that includes (i) a power input element configured to connect with and receive power from a power source; (ii) a power adjustment device connected to said power input element optionally comprising a data input element connected to a data processing device; (iii) a power output element connected to said power adjustment device and configured to connect with and provide power to a processing element; optionally (iv) a data output element connected to the data processing device and connected to said power output element; wherein said power output element is configured to be controlled by said power adjustment device and wherein said controlling device is configured to be readily removable from the power source and the processing element.

### CROSS-REFERENCE TO RELATED APPLICATIONS

The instant application is a continuation-in-part of International Patent Application No. PCT/IB2017/052065 filed Apr. 10, 2017 and published as WO2017/178958 on Oct. 19, 2017, and claims priority under 35 U.S.C. § 119(e) of U.S. provisional Patent Application No. 62/320,541 filed on Apr. 10, 2016. The disclosure of each of these applications is expressly incorporated by reference herein in its entirety.

### BACKGROUND

There is an answered need for controlled administering to a patient a therapeutic composition, particularly when in vapors or spray form. The problem is even greater in cases where the therapeutic composition comprises multiple therapeutically active components. There is also a need for improving treatment data collection and processing.

### SUMMARY OF THE INVENTION

According to a first aspect, provided is a controlling device particularly adapted for vaporizing and/or spraying a therapeutic composition, comprising (i) a power input element configured to connect with and receive power from a power source; (ii) a power adjustment device connected to said power input element, (iii) a power output element connected to said power adjustment device and configured to connect with and provide power to a processing element; and optionally (iv) a power-storing device configured to couple with said power input element, and optionally (v) an electric transformer configured to couple with said power source, and wherein said power output element is configured to be controlled by said power adjustment device. According to an embodiment, said controlling device is configured to be readily removable from the power source and the processing element.

According to an embodiment, said processing element is configured to connect to said controlling device and comprises a controllable unit selected from the group consisting of a heating unit, a gas or liquid input opening, an additive input opening and combinations thereof and said controllable unit is configured to receive power from said power output element

According to an embodiment, further provided is an article for administering a therapeutic composition by vaporizing and/or spraying comprising said controlling device and a processing element, wherein said processing element comprises a chamber and a heating unit set up to heat said chamber and/or the content of said chamber and wherein said chamber comprises an opening designed for input of said therapeutic composition, an opening for output of vapors, or both.

According to a second aspect, provided is a controlling device particularly adapted for administering a therapeutic composition, comprising (i) a power input element configured to connect to and receive power from a power source; (ii) a data input element connected to the power input element and to a data processing device; (iii) a power output element connected to said data input element and configured to connect with and provide power to a processing element; optionally (iv) a data output element connected to the data processing device and connected to said power output element; optionally (vi) a power-storing device configured to couple with said power input element, and optionally (vii) an electric transformer configured to couple with a power source, and, wherein said power output element is configured to be controlled by said data input element. According to an embodiment, said controlling device includes a power adjustment device connected to the data output element and the power output element.

According to an embodiment, said administering comprises at least one of vaporizing and spraying.

According to an embodiment, said processing element comprises a controllable unit selected from a heating unit, a gas or liquid input opening, an additive input opening and combinations thereof and said controllable unit is configured to receive power from said power output element.

According to an embodiment, further provided is an article for administering a therapeutic composition by vaporizing and/or spraying, comprising said controlling device and a processing element, wherein said processing element comprises a chamber and a heating unit set up to heat said chamber and/or the content of said chamber and wherein said chamber comprises an opening designed for input of said therapeutic composition, an opening for output of vapors, or both.

According to a third aspect, provided is an article particularly adapted for administering to a patient a therapeutic composition by vaporizing, comprising (i) a vaporizing element configured to vaporize said therapeutic composition and optionally to activate the therapeutic composition; (ii) a power input element configured to connect to and receive power from a power source; (ii) a data input element connected to the power input element and to a data processing device; (iii) a power output element connected to said data input element and configured to connect with and provide power to said vaporizing element; optionally (iv) a data output element connected to the data processing device and connected to said power output element; optionally (vi) a power-storing device configured to couple with said power input element, and optionally (vii) an electric transformer configured to couple with a power source, and, wherein said power output element is configured to be controlled by said data input element. According to an embodiment, said article includes a power adjustment device connected to the data output element and the power output element.

According to an embodiment, said vaporizing element comprises a controllable heating unit, which heating unit is configured to be powered by said power output. According to an embodiment, said vaporizing element comprises a vaporizing chamber and a heating unit configured to heat said chamber and/or the content thereof, which heating unit is configured to be powered by said power output.

According to an embodiment, said vaporizing element comprises at least one of a display and a temperature measurement device, and said temperature measurement device is configured to provide data to said data processing device, optionally via said data output element. According to an embodiment, said vaporizing element comprises a sensor, and said sensor is configured to provide data to said data processing device, optionally via said data output element.

According to an embodiment, said vaporizing chamber comprises an input opening, which opening is configured to receive said therapeutic composition from a package containing said therapeutic composition. According to an embodiment, said package containing said therapeutic composition includes processing parameters data and wherein said data processing device is configured to read said processing parameters data. According to an embodiment, said data processing device is configured to control said power output according to said processing parameters and according to patient personal data.

According to an embodiment, the data output element is configured to provide data to said data processing device, optionally via said data output element, and said data is selected from the group consisting of processing parameters, vaporizing dates, vaporizing hours, connecting and/or disconnecting data from said power source, connecting and/or disconnecting data from said vaporizing element and combinations thereof.

According to an embodiment, said data processing device is configured to store data received from said data output element and patient personal data. According to an embodiment, said data processing device is configured to process said received and stored data. According to an embodiment, said data processing device is configured to share said processed data with a central medical records processing device.

According to an embodiment, said vaporizing chamber comprises an output opening configured to connect with a vessel designed to store formed vapors. According to an embodiment, said vaporizing chamber comprises a gas or liquid input opening configured to introduce a gas or a liquid to said vaporizing unit. According to an embodiment, wherein said vaporizing chamber gas or liquid input opening is configured to be controlled by said data processing unit.

According to an embodiment, said vaporizing chamber comprises an input opening configured to receive said therapeutic composition from a package containing said therapeutic composition and said vaporizing chamber further comprises a gas or a liquid input opening, which gas or liquid input opening is configured to introduce a gas a or liquid to said vaporizing unit, and said package containing said therapeutic composition includes processing parameters data, and said data processing device is configured to read said processing parameters data and said data processing device is configured to control said gas or a liquid input opening according to said processing parameters and according to patient personal data.

According to an embodiment, said vaporizing chamber comprises an additive input opening configured to introduce an additive to said vaporizing unit. According to an embodiment, said vaporizing chamber additive input opening is configured to be controlled by said data processing unit.

According to a fourth aspect, provided is an article particularly adapted for administering to a patient a therapeutic composition by spraying, comprising (i) a spraying element configured to spray said therapeutic composition and optionally to activate the therapeutic composition; (ii) a power input element configured to connect to and receive power from a power source; (ii) a data input element connected to the power input element and to a data processing device; (iii) a power output element connected to said data input element and configured to connect with and provide power to said spraying element; optionally (iv) a data output element connected to the data processing device and connected to said power output element; optionally (vi) a power-storing device configured to couple with said power input element, and optionally (vii) an electric transformer configured to couple with a power source, and, wherein said power output element is configured to be controlled by said data input element. According to an embodiment, said spraying element comprises a heating unit, which heating unit is configured to be powered by said power output element.

According to an embodiment, said spraying element comprises at least one of a display and a flow measurement device, which flow measurement device is configured to provide data to said data processing device, optionally via said data output element. According to an embodiment, said spraying element comprises a sensor, and wherein said sensor is configured to provide data to said data processing device, optionally via said data output element.

According to an embodiment, said spraying element comprises an input opening, which opening is configured to receive a sprayable composition from a package containing said therapeutic composition. According to an embodiment, said package containing said therapeutic composition includes processing parameters data and wherein said data processing device is configured to read said processing parameters data. According to an embodiment, said data processing device is configured to control said power output element according to said processing parameters and according to patient personal data.

According to an embodiment, the data output element is configured to provide data to said data processing device, optionally via said data output element, and which data is selected from the group consisting of processing parameters, spraying dates, spraying hours, connecting and/or disconnecting data from said power source, connecting and/or disconnecting data from said spraying element and combinations thereof.

According to an embodiment, said data processing device is configured to store data received from said data output element and patient personal data. According to an embodiment, said data processing device is configured to process said received and stored data. According to an embodiment, said data processing device is configured to share said processed data with a central medical records processing device.

According to an embodiment, said spraying element comprises a gas or a liquid input opening configured to introduce gas or a liquid to said spraying element. According to an embodiment, said spraying element gas or liquid input opening is configured to be controlled by said data processing unit.

According to an embodiment, said spraying element comprises an additive input opening configured to introduce an additive to said spraying element. According to an embodiment, said spraying element additive input opening is configured to be controlled by said data processing unit noptionally via said data output element.

According to an embodiment, further provided is a method for the controlled administering of a therapeutic composition to a patient, comprising: (i) providing said therapeutic composition; (ii) collecting processing parameters input data and storing said collected input data on a data processing device; (iii) introducing said therapeutic composition into a processing element; and (iv) processing said therapeutic composition in said processing element to form vapors or a spray comprising said therapeutic composition; wherein said processing is powered by an adjustable power source and wherein said adjustable power source is controlled by said data processing device.

According to an embodiment, said therapeutic composition comprises at least one of a cannabinoid, a solution of a cannabinoid in a solvent, a cannabis plant material, a cannabis extract and a solution of said extract in a solvent. According to an embodiment, said therapeutic composition comprises an activated cannabinoid.

According to an embodiment, said method further comprises providing said therapeutic composition in a package including said processing parameters input data which is collected and stored. According to an embodiment, said method further comprises collecting operating parameters output data and storing said collected output data on a data processing device.

According to an embodiment, said operating parameters output data is selected from the group consisting of operating parameters, vaporization dates, vaporization hours, connecting and/or disconnecting data from said power source, connecting and/or disconnecting data from said vaporizing element and combinations thereof.

According to an embodiment, said method further comprises processing said collected output data on a data processing device including other patient personal data. According to an embodiment, said method further comprises sharing said processed data with a central medical records processing device.

According to an embodiment, said processing comprises evaporating and optionally activating in a processing element comprising a vaporizing chamber and a heating unit configured to heat said chamber and/or the content thereof, wherein such heating is powered by said power output.

According to an embodiment, said method further comprises introducing a gas or a liquid into said vaporizing chamber through a gas or a liquid input opening controlled by said data processing unit. According to an embodiment, said method further comprises introducing an additive into said vaporizing chamber through an additive input opening controlled by said data processing unit. According to an embodiment, said additive comprises a terpene.

According to an embodiment, said processing comprises spraying in a spraying chamber, which spraying is powered by said power output.

According to an embodiment, said method further comprises introducing an additive into said spraying chamber through an additive input opening controlled by said data processing unit. According to an embodiment, said additive comprises a terpene.

### BRIEF DESCRIPTION OF THE DRAWINGS

FIG. 1 schematically shows an exemplary controlling device particularly adapted for administering a therapeutic composition, in accordance with exemplary embodiment of present invention;

FIG. 2 schematically shows an exemplary controlling device particularly adapted for administering a therapeutic composition attached to a vaporizing element or a spraying element, in accordance with exemplary embodiment of present invention;

FIG. 3 schematically shows an addition exemplary controlling device particularly adapted for administering a therapeutic composition, in accordance with exemplary embodiment of present invention;

FIG. 4 schematically shows an addition exemplary controlling device particularly adapted for administering a therapeutic composition attached to a vaporizing element or a spraying element, in accordance with exemplary embodiment of present invention;

FIG. 5 schematically shows an exemplary vaporizing element or a spraying element that can be used in with exemplary embodiment of present invention; and

FIG. 6 schematically shows an exemplary vaporizing element or a spraying element that can be used in with exemplary embodiment of present invention while the power element is disconnected.

### DETAILED DESCRIPTION OF THE INVENTION

### Definitions

As used herein the term “therapeutic composition” refers to a composition capable of relieving a medical situation, e.g. illness, pain and to a composition capable of relieving conditions resulting from a medical treatment.

As used herein the term “therapeutically active component” refers to a component in a therapeutic composition, which component is capable of relieving a medical situation, e.g. illness, pain and to a component capable of relieving conditions resulting from a medical treatment.

As used herein the term “therapeutic cannabis composition” refers to a therapeutic composition comprising a cannabis plant component.

As used herein the term “therapeutic cannabis component” refers to cannabinoids, terpenes or mixtures thereof.

As used herein the terms vaporize, vaporizing and vaporization refer to conversion of a solid or a liquid compound into a vapor form, e.g. as in boiling or sublimation. As used herein the term vaporizable refers to a compound, e.g. a therapeutic compound which can be vaporized at a temperature under 300° C., while at atmospheric pressure.

As used herein the terms “vaporizer pen” and “vape pen” refer to a commercially available device used for generating vapors for inhalation.

As used herein the terms spraying and spray refer to conversion of a solid or a liquid compound into particles small enough to be carried in a carrier gas, e.g. air, e.g. as in an aerosol. As used herein the term sprayable refers to a compound, e.g. a therapeutic compound which can be sprayed.

As used herein, the term power refers to electrical power, the term power supply refers to supply of electric power and the term power source refers to a source of electric power. As used herein, the term transformer refers to an electric transformer.

As used herein, the terms “data processing” and “data processing device” refer also to “data storing” and to “data storing device”, respectively.

As used herein, the term “processing parameters” refers to parameters determining the way of processing, e.g. instructions or recommendations for how to process. For example, vaporization processing parameters include instructions or recommendations for vaporization temperature, vaporization duration in a vaporization chamber and optionally also for rate of air flow through such chamber.

As used herein, the term “operating parameters” refers to parameters actually used during the operation, e.g. parameters actually used during the processing. For example, vaporization operating parameters include actual vaporization temperature, actual vaporization and optionally also actual rate of air flow through such the vaporization chamber.

As used herein, the term “activating” refers to conversion of a compound from its less active or inactive form into the active form. For example, cannabinoids typically appear in cannabis plant material or its extract in their acid form (at least partially). Thermal treatment leads to decarboxylation, i.e. removal of the acid function. When referring to cannabinoids, the terms “activating” and “activation” refer to decarboxylation and the term “activated component” refers to decarboxylated cannabinoid.

Unless indicated otherwise, percent is weight percent and ratio is weight/weight ratio.

The particulars shown herein are by way of example and for purposes of illustrative discussion of the various embodiments of the present invention only and are presented in the cause of providing what is believed to be the most useful and readily understood description of the principles and conceptual aspects of the invention. In this regard, no attempt is made to show details of the invention in more detail than is necessary for a fundamental understanding of the invention, the description making apparent to those skilled in the art how the several forms of the invention may be embodied in practice.

The present invention will now be described by reference to more detailed embodiments. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. As used in the description of the invention and the appended claims, the singular forms “a,” “an,” and “the” are intended to include the plural forms as well, unless the context clearly indicates otherwise.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Additional advantages of the invention will be set forth in part in the description which follows, and in part will be obvious from the description, or may be learned by practice of the invention. It is to be understood that both the foregoing general description and the following detailed description are exemplary and explanatory only and are not restrictive of the invention, as claimed.

### First Aspect of Embodiments

According to a first aspect, provided is a controlling device particularly adapted for vaporizing and/or spraying a therapeutic composition, comprising (i) a power input element configured to connect with and receive power from a power source; (ii) a power adjustment device connected to said power input element, (iii) a power output element connected to said power adjustment device and configured to connect with and provide power to a processing element; and optionally (iv) a power-storing device configured to couple with said power input element, and optionally (v) an electric transformer configured to couple with said power source, and wherein said power output element is configured to be controlled by said power adjustment device.

According to an embodiment, said therapeutic composition is vaporizable. According to an embodiment, said therapeutic composition is sprayable.

According to an embodiment, said power source is a battery.

According to an embodiment, said processing element is configured to receive said therapeutic composition and to emit vapors or a spray of said therapeutic composition.

According to an embodiment, said power input element, said power output element, or both, are selected from the group consisting of a socket, a USB connector, a standard vaporizer pen screw and combinations thereof.

According to an embodiment, said power-storing device is selected from the group consisting of battery, capacitor and combinations thereof.

According to an embodiment of said first aspect, said processing element is configured to connect to said controlling device and comprises a controllable unit selected from the group consisting of a heating unit, a gas or liquid input opening, an additive input opening and combinations thereof and said controllable unit is configured to receive power from said power output element.

According to an embodiment, said controllable heating unit is a vaporizing pen cartridge.

According to an embodiment further provided is an article for administering a therapeutic composition by vaporizing and/or spraying, comprising said controlling device, and a processing element, wherein said processing element comprises a chamber and a heating unit set up to heat said chamber and/or the content of said chamber and wherein said chamber comprises an opening designed for input of said therapeutic composition, an opening for output of vapors, or both.

### Second Aspect

According to a second aspect, provided is a controlling device particularly adapted for administering a therapeutic composition, comprising (i) a power input element configured to connect to and receive power from a power source; (ii) a data input element connected to the power input element and to a data processing device; (iii) a power output element connected to said data input element and configured to connect with and provide power to a processing element; optionally (iv) a data output element connected to the data processing device and connected to said power output element; optionally (vi) a power-storing device configured to couple with said power input element, and optionally (vii) an electric transformer configured to couple with a power source, and, wherein said power output element is configured to be controlled by said data input element.

According to an embodiment, said controlling device includes a power adjustment device connected to the data output element and the power output element.

According to an embodiment, said therapeutic composition is vaporizable. According to an embodiment, said therapeutic composition is sprayable.

According to an embodiment, said processing comprises at least one of vaporizing and spraying.

According to an embodiment, said data processing device comprises at least one of CPU, ROM, RAM and microchip.

According to an embodiment, said data input element, said data output element or both are selected from wire communication, wireless communication and combination thereof.

According to an embodiment, said wireless communication is configured to use a wireless protocol, e.g. selected from the group consisting of UPB, INSTEON, Z-Wave, Bluetooth, HomePlug, INSTEON, Wi-Fi and ZigBee.

According to an embodiment, said power source is a battery. According to an embodiment, said power source is the battery of said data processing device.

According to an embodiment, said processing element is configured to receive said therapeutic composition and to emit vapors of said therapeutic composition.

According to an embodiment, said power input element, said power output element, or both, are selected from the group consisting of a socket, a USB connector, a standard vaporizer pen screw and combinations thereof.

According to an embodiment, said power-storing device is selected from the group consisting of battery, capacitor and combinations thereof.

According to an embodiment of said second aspect, said processing element comprises a controllable unit selected from a heating unit, a gas or liquid input opening, an additive input opening and combinations thereof and said controllable unit is configured to receive power from said power output element.

According to an embodiment, said heating unit is a vaporizing pen cartridge.

According to an embodiment further provided is an article for administering a therapeutic composition by vaporizing and/or spraying comprising said controlling device, and a processing element, wherein said processing element comprises a chamber and a heating unit set up to heat said chamber and/or the content of said chamber and said chamber comprises an opening designed for input of said therapeutic composition, an opening for output of vapors, or both.

### Third Aspect

According to a third aspect, provided is an article particularly adapted for administering to a patient a therapeutic composition by vaporizing, comprising (i) a vaporizing element configured to vaporize said therapeutic composition and optionally to activate the therapeutic composition; (ii) a power input element configured to connect to and receive power from a power source; (ii) a data input element connected to the power input element and to a data processing device; (iii) a power output element connected to said data input element and configured to connect with and provide power to said vaporizing element; optionally (iv) a data output element connected to the data processing device and connected to said power output element; optionally (vi) a power-storing device configured to couple with said power input element, and optionally (vii) an electric transformer configured to couple with a power source, and, wherein said power output element is configured to be controlled by said data input element.

According to an embodiment, said article includes a power adjustment device connected to the data output element and the power output element.

According to an embodiment, said therapeutic composition is vaporizable. According to an embodiment, said therapeutic composition is sprayable.

According to an embodiment, said data processing device comprises at least one of CPU, ROM, RAM and microchip.

According to an embodiment, said data input element, said data output element or both are selected from wire communication, wireless communication and combination thereof.

According to an embodiment, said wireless communication is configured to use a wireless protocol, e.g. selected from the group consisting of UPB, INSTEON, Z-Wave, Bluetooth, HomePlug, INSTEON, Wi-Fi and ZigBee.

According to an embodiment, said power source is a battery. According to an embodiment, said power source is the battery of said data processing device.

According to an embodiment, said vaporizing element is configured to receive said therapeutic composition and to emit vapors of said therapeutic composition.

According to an embodiment, said power input element, said power output element, or both, are selected from the group consisting of a socket, a USB connector, a standard vaporizer pen screw and combinations thereof.

According to an embodiment, said power-storing device is selected from the group consisting of battery, capacitor and combinations thereof.

According to an embodiment, said vaporizing element comprises a controllable heating unit, which heating unit is configured to be powered by said power output.

According to an embodiment, said vaporizing element comprises a vaporizing chamber and a heating unit configured to heat said chamber and/or the content thereof, which heating unit is configured to be powered by said power output.

According to various embodiment, said vaporizing chamber is composed of glass, metal, plastic or a combination thereof. According to various embodiment, the volume of said vaporizing chamber is between 1 milliliter and 20 milliliter.

According to an embodiment, said vaporizing element comprises at least one of a display and a temperature measurement device. According to an embodiment, said temperature measurement device is configured to provide data to said data processing device, optionally via said data output element According to an embodiment, said vaporizing element comprises flow meter. According to an embodiment, said flow meter is configured to provide data to said data processing device, optionally via said data output element. According to an embodiment, said vaporizing element comprises a sensor, and wherein said sensor is configured to provide data to said data processing device, optionally via said data output element.

According to an embodiment, said vaporizing chamber comprises an input opening, which opening is configured to receive said therapeutic composition from a package containing said therapeutic composition. According to an embodiment, said package containing said therapeutic composition includes processing parameters data and said data processing device is configured to read said processing parameters data. According to an embodiment, said vaporizing parameters data comprises at least one of vaporization temperature, heating time and flow rate of input gas. According to an embodiment, said data processing device is configured to control said power output according to said processing parameters and according to patient personal data. According to an embodiment, said patient personal data comprises at least one of patient health and medical condition and/or indication, patient age, body weight gender, patient diet, patient eating schedule, patient received medication, patient treatment history and instructions from treating personnel. According to an embodiment, said data processing device is configured to block processing or limit it as required, e.g. by the history of administrating or by instruction of a treating personnel, e.g. a physician.

According to an embodiment, said article comprises a data output element configured to provide data to said data processing device, optionally via said data output element. According to an embodiment, data provided to said data processing device is selected from the group consisting of operating parameters, vaporization dates, vaporization hours, connecting and/or disconnecting data from said power source, connecting and/or disconnecting data from said vaporizing element and combinations thereof. According to an embodiment, said data processing device is configured to store data received from said data output element, vaporization dates, vaporization hours and patient personal data. According to an embodiment, said patient personal data comprises at least one of patient health and medical condition and/or indication, patient age, body weight. patient gender, patient diet, patient eating schedule, patient received medication and patient treatment history. According to an embodiment, said data processing device is configured to process said received and stored data. According to an embodiment, said data processing device is configured to share said stored data and said processed data with a central medical records processing device.

According to an embodiment, said vaporizing chamber comprises an output opening configured to connect with a vessel designed to store formed vapors. According to an embodiment, said output opening comprises a flow meter configured to measure the rate of vapors flow into said vapors storing vessel. According to an embodiment, said device comprises a data output element configured to provide data from said flow meter to said data processing device.

According to an embodiment, said vaporizing chamber comprises a gas or a liquid input opening configured to introduce a gas, e.g. air, or a liquid to said vaporizing unit. According to an embodiment, said gas or liquid input opening is configured to be controlled by said data processing unit. According to an embodiment, said device comprises a data output element configured to provide data from said gas or liquid input opening to said data processing device, e.g. time of opening, duration of opening and flow rate through said opening.

According to an embodiment, said vaporizing chamber comprises an input opening configured to receive said therapeutic composition from a package containing said therapeutic composition and said vaporizing chamber further comprises a gas or liquid input opening, which gas or liquid input opening is configured to introduce a gas or a liquid into said vaporizing unit, said package containing said therapeutic composition includes processing parameters data, said data processing device is configured to read said processing parameters data and said data processing device is configured to control said gas or liquid input opening according to said processing parameters and according to patient personal data.

According to an embodiment, said vaporizing chamber comprises an additive input opening configured to introduce an additive to said vaporizing unit. According to an embodiment, said vaporizing chamber additive input opening is configured to be controlled by said data processing unit. According to an embodiment, said device comprises a data output element configured to provide data from said additive input opening to said data processing device, e.g. time of opening, duration of opening and the amount of added additive.

### Fourth Aspect

According to a fourth aspect, provided is an article particularly adapted for administering to a patient a therapeutic composition by spraying, comprising (i) a spraying element configured to spray said therapeutic composition and optionally to activate the therapeutic composition; (ii) a power input element configured to connect to and receive power from a power source; (ii) a data input element connected to the power input element and to a data processing device; (iii) a power output element connected to said data input element and configured to connect with and provide power to said spraying element; optionally (iv) a data output element connected to the data processing device and connected to said power output element; optionally (vi) a power-storing device configured to couple with said power input element, and optionally (vii) an electric transformer configured to couple with a power source, and, wherein said power output element is configured to be controlled by said data input element.

According to an embodiment, said article includes a power adjustment device connected to the data output element and the power output element.

According to an embodiment, said data processing device comprises at least one of CPU, ROM, RAM and microchip.

According to an embodiment, said data input element, said data output element or both are selected from wire communication, wireless communication and combination thereof. According to an embodiment, said wireless communication is configured to use a wireless protocol, e.g. selected from the group consisting of UPB, INSTEON, Z-Wave, Bluetooth, HomePlug, INSTEON, Wi-Fi and ZigBee.

According to an embodiment, said power source is a battery. According to an embodiment, said power source is the battery of said data processing device.

According to an embodiment, said spraying element is configured to receive said therapeutic composition and to emit spray of said therapeutic composition.

According to an embodiment, said power input element, said power output element, or both, are selected from the group consisting of a socket, a USB connector, a standard vaporizer pen screw and combinations thereof.

According to an embodiment, said power-storing device is selected from the group consisting of battery, capacitor and combinations thereof.

According to an embodiment, said spraying element comprises a controllable heating unit, which heating unit is configured to be powered by said power output.

According to an embodiment, said spraying element comprises a spraying chamber.

According to an embodiment, said spraying element comprises at least one of a display and a flow measurement device, which flow measurement device is configured to provide data to said data processing device, optionally through said data output element. According to an embodiment, said spraying element, comprises a sensor, and said sensor is configured to provide data to said data processing device, optionally via said data output element.

According to an embodiment, said spraying chamber comprises an input opening, which opening is configured to receive said therapeutic composition from a package containing said therapeutic composition. According to an embodiment, said package containing said therapeutic composition includes processing parameters data and said data processing device is configured to read said processing parameters data. According to an embodiment, said data processing device is configured to control said power output according to said processing parameters and according to patient personal data. According to an embodiment, said patient personal data comprises at least one of patient health and medical condition and/or indication, patient age, body weight, patient gender, patient diet, patient eating schedule, patient received medication, patient treatment history and instructions from treating personnel. According to an embodiment, said data processing device is configured to block processing or limit it as required, e.g. by the history of administrating or by instruction of a treating person, e.g. a physician.

According to an embodiment, said device comprises a data output element configured to provide data to said data processing device, which data comprises data selected from the group consisting of processing parameters, spraying dates and spraying hours connecting and/or disconnecting data from said power source, connecting and/or disconnecting data from said spraying element and combinations thereof.

According to an embodiment, said data processing device is configured to store data received from said data output element and patient personal data. According to an embodiment, said patient personal data comprises at least one of patient health and medical condition and/or indication, patient age, body weight, patient gender, patient diet, patient eating schedule, patient received medication and patient treatment history. According to an embodiment, said data processing device is configured to process said received and stored data. According to an embodiment, said data processing device is configured to share said stored data and said processed data with a central medical records processing device.

According to an embodiment, said spraying chamber comprises a gas or liquid input opening configured to introduce a gas, e.g. air, or a liquid to said spraying unit. According to an embodiment, said gas or liquid input opening is configured to be controlled by said data processing unit. According to an embodiment, said device comprises a data output element configured to provide data from said gas or liquid input opening to said data processing device, e.g. time of opening, duration of opening and flow rate through said opening.

According to an embodiment, said spraying chamber comprises an input opening configured to receive said therapeutic composition from a package containing said therapeutic composition and said spraying chamber further comprises a gas or liquid input opening, which gas or liquid input opening is configured to introduce a gas or liquid to said spraying unit, said package containing said therapeutic composition includes processing parameters data, said data processing device is configured to read said processing parameters data and said data processing device is configured to control said gas or liquid input opening according to said processing parameters and according to patient personal data.

According to an embodiment, said spraying chamber comprises an additive input opening configured to introduce an additive to said spraying unit. According to an embodiment, said spraying chamber additive input opening is configured to be controlled by said data processing unit. According to an embodiment, said device comprises a data output element configured to provide data from said additive input opening to said data processing device, e.g. time of opening, duration of opening and the amount of added additive.

### Fifth Aspect

According to a fifth aspect, provided is a method for controlled administrating to a patient a therapeutic composition, comprising: (i) providing said therapeutic composition; (ii) collecting processing parameters input data and storing said collected input data on a data processing device; (iii) introducing said therapeutic composition into a processing element; and (iv) processing said therapeutic composition in said processing element to form vapors or a spray comprising said therapeutic composition; wherein said processing is powered by an adjustable power source and wherein said adjustable power source is controlled by said data processing device.

According to an embodiment, said provided therapeutic composition is vaporizable. According to an embodiment, said provided therapeutic composition is sprayable. According to an embodiment, said provided therapeutic composition is in a solid form. According to an alternative embodiment, said provided therapeutic composition is in a liquid form.

According to an embodiment, said therapeutic composition comprises multiple active components. According to an embodiment, said therapeutic composition is therapeutic cannabis composition. According to an embodiment, said therapeutic composition comprises therapeutic cannabis component. According to an embodiment, said therapeutic composition comprises therapeutic cannabis component and a pharmaceutical compound.

According to an embodiment, said provided therapeutic composition comprises at least one of a cannabinoid, a solution of a cannabinoid in a solvent, a cannabis plant material, a cannabis extract and a solution of said extract in a solvent.

According to an embodiment, said cannabinoid is selected from the group consisting of acid or decarboxylated forms of tetrahydrocannabidiol (THC), cannabidiol (CBD), cannabigerol (CBG), cannabichromene (CBC) tetrahydrocannabivarin (THCV) Cannabidivarin (CBDV), cannabinol (CBN) and combinations thereof.

According to an embodiment, said provided therapeutic composition comprises an activated cannabinoid, e.g. a decarboxylated cannabinoids. According to an embodiment, said providing comprises thermal treatment at a temperature and for a time sufficient to decarboxylate at least 80% of said cannabinoid, at least 90% or at least 95%. According to an alternative embodiment, at least a fraction of said cannabinoid is in a non-activated form, e.g. in its acid form, and said processing comprises activation.

According to an embodiment, said provided therapeutic composition comprises a solution of a cannabinoid in a solvent. According to an embodiment, said solvent is selected from the group consisting of vegetable oil, terpenes, plant extracts and combinations thereof. According to an embodiment, said cannabis plant material comprises cannabis flowers. According to an embodiment, said providing comprises drying cannabis plant material, e.g. flowers, to form said therapeutic composition. According to an embodiment, said therapeutic composition comprises a cannabis extract. According to an embodiment, said providing comprises extracting cannabis plant material, e.g. flowers, to form said therapeutic composition. According to an embodiment, said extracting comprises using an extractant selected from the group consisting of alcohols, e.g. ethanol, super-critical CO₂, sub-critical CO₂, near-critical CO₂, and combinations thereof.

According to an embodiment, said method further comprises providing said therapeutic composition in a package comprising said processing parameters input data to be collected and stored, e.g. in the form of a barcode. According to an embodiment, said processing parameters include at least one of processing temperature, processing duration (as such or per unit weight or volume of the therapeutic composition) and flow rate of air through said processing element.

According to an embodiment, said adjustable power source is controlled by said data processing device according to said processing parameters and according to patient personal data. According to an embodiment, said patient personal data comprises at least one of patient health and medical condition and/or indication, patient age, body weight and gender, patient diet, patient eating schedule, patient received medication, patient treatment history and instructions from treating personnel. According to an embodiment, said data processing device is configured to block processing or limit it as required, e.g. by the history of administrating or by instruction of a treating person, e.g. a physician.

According to an embodiment, said method further comprises collecting operating parameters output data and storing said collected output data on a data processing device. According to an embodiment, said operating parameters output data is selected from the group consisting of operating parameters, vaporization dates, vaporization hours, connecting and/or disconnecting data from said power source, connecting and/or disconnecting data from said vaporizing element and combinations thereof. According to an embodiment, said operating parameters include at least one of processing temperature, processing duration (as such or per unit weight or volume of the therapeutic composition) and flow rate of air through said processing element.

According to an embodiment, said method further comprises processing said collected output data on a data processing device along with other patient personal data. According to an embodiment, said patient personal data comprises at least one of patient health and medical condition and/or indication, patient age, body weight and gender, patient diet, patient eating schedule, patient received medication and patient treatment history. According to an embodiment, said method further comprises sharing said processed data with a central medical records processing device.

According to an embodiment, said processing comprises evaporating and optionally activating in a processing element comprising a vaporizing chamber and a heating unit configured to heat said chamber and/or the content thereof; which heating unit is powered by said power output.

According to an embodiment, said method further comprises introducing a gas or liquid into said vaporizing chamber through a gas or liquid input opening controlled by said data processing unit. According to an embodiment, said gas is air.

According to an embodiment, said method further comprises introducing an additive into said vaporizing chamber through an additive input opening controlled by said data processing unit. According to an embodiment, said additive comprises a terpene. According to an embodiment, said provided therapeutic composition comprises at least one of a cannabinoid, a solution of a cannabinoid in a solvent, a cannabis plant material, a cannabis extract and a solution of said extract in a solvent and said additive comprises a terpene, e.g. a terpene present in a cannabis plant at a concentration greater than 0.01%.

According to an embodiment, said processing comprises spraying in a spraying chamber, which spraying is powered by said power output.

According to an embodiment, said method further comprises introducing an additive into said spraying chamber through an additive input opening controlled by said data processing unit. According to an embodiment, said additive comprises a terpene. According to an embodiment, said provided therapeutic composition comprises at least one of a cannabinoid, a solution of a cannabinoid in a solvent, a cannabis plant material, a cannabis extract and a solution of said extract in a solvent and said additive comprises a terpene′ e.g. a terpene present in a cannabis plant at a concentration greater than 0.01%.

The power source is of course not part of the controlling device, which typically begins with the power input element. Power storing and electric transformer are part of the controlling device so typically come after the power input element. The data processing device, in most cases, is not part of the controlling device, but external to it (e.g. a cell phone). And the processing element is not part of the controlling device, but is part of the overall functioning article.

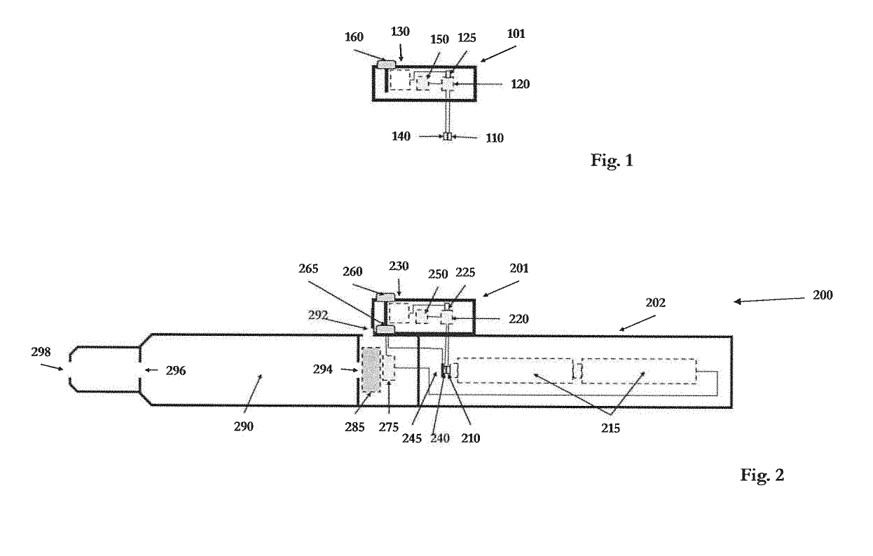

FIG. 1 shows an exemplary controlling device 101 particularly adapted for controlling the administration of a therapeutic composition via a vaporizing or a spraying element that comprises a power input element 110 configured to connect with and receive power from a power source. A power adjustment device 120 is connected to the power input element 110 as well as to an optional data input element 125 and a data processing device 130. The data input element 110 and the data processing device 130 are connected. A power output element 140 is connected to the power adjustment device 120 and is configured to connect with and provide power to the vaporizing or spraying element 101 when connected. An optional power-storing device 150 is configured to couple with the power input element 110. The exemplary controlling device 101 also includes an operating button 160 that can activate the operating button of the processing element when the unit 101 is connected to a vaporizing element or a spraying element.

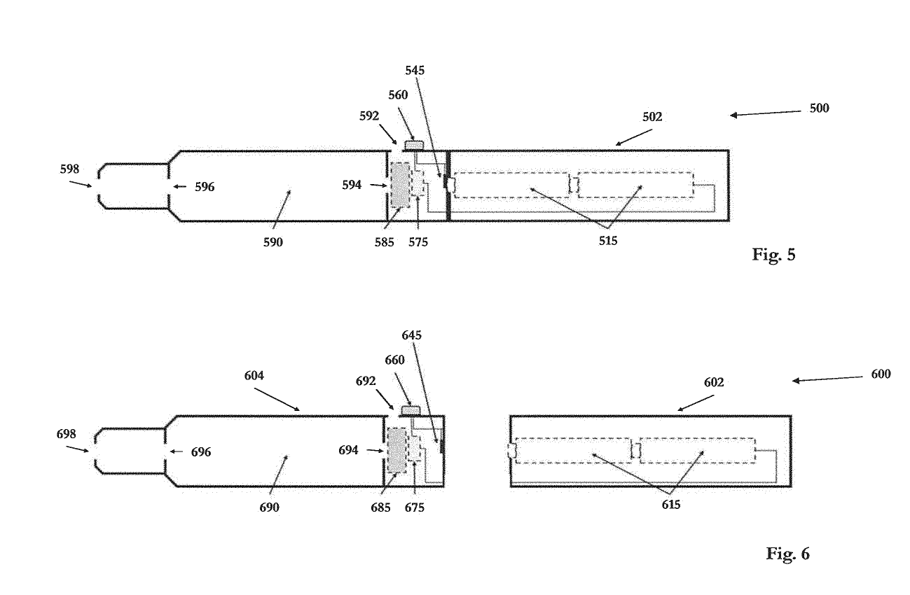

FIG. 2 shows an exemplary controlling device 201 (which can be of the same type as shown in FIG. 1) attached and/or connected to a vaporizing element or a spraying element 202 for controlling administering a therapeutic composition. When unit 201 is connected to unit 202, they form a unit 200. The vaporizing element or spraying element 202 comprises a power source 215, an operating button 265, and a heating element 275 (normally powered by the power source 215 by the operating button 265), and is configured to heat the package 285 containing therapeutic composition. The vaporizing element or a spraying element 202 further includes a vaporizing chamber 290, and an aspiration through opening 298. When operating button 265 is pressed, air is caused to pass through air intake 292 and to be mixed with therapeutic composition held in package 285 containing therapeutic composition (which is heated by heating element 275) and enters the vaporizing chamber or the spraying element via opening 294. The mixture then passes via opening 296 and is aspired via opening 298. The controlling device 201 further utilizes a power input element 210 configured to connect with and receive power from a power source 215. A power adjustment device 220 is connected to the power input element 210 and also optionally to a data input element 225 connected to a data processing device 230. The controlling device 201 further utilizes a power output element 240 configured to be controlled by the power adjustment device 220 and to provide power to the power connecting element 245. An optional power-storing device 250 is configured to couple with the power input element 210. An optional electric transformer (not shown) is configured to couple with a power source. The power output element 240 is configured to be controlled by the power adjustment device 220. The controlling device 201 is configured to be readily removable from the power source 215 and the processing element. The exemplary controlling device 201 also includes an operating button 260 that can be pressed by a user in order to activate the operating button 265. One can attach or connect, e.g., removably connect, the controlling device 201 to the vaporizing element or a spraying element 202. The power source 215 is connected to the heating element 275 which is controlled by to the controlling device 201. This exemplary controlling device 201 can also be fitted to a vaporizing element or a spraying element with internal power source of the type shown FIG. 5.

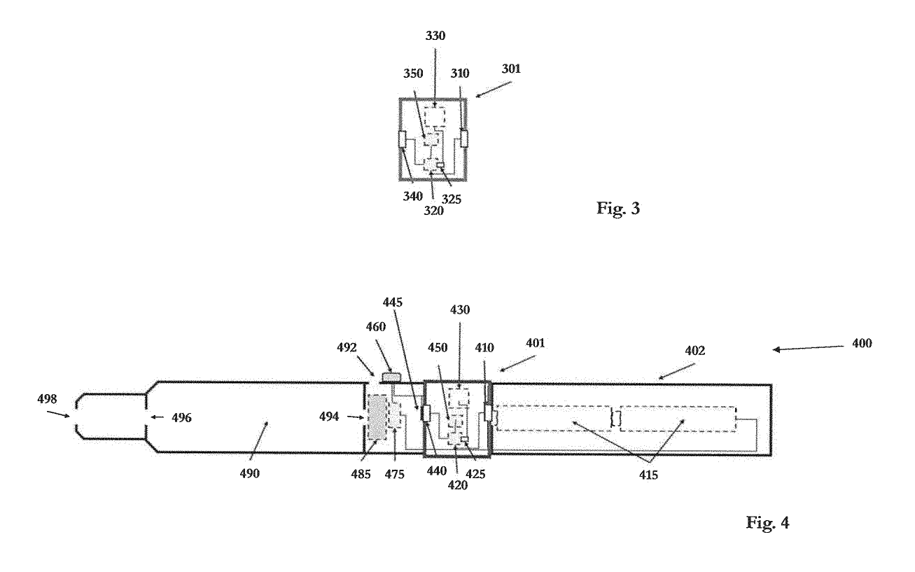

FIG. 3 shows an exemplary controlling device 301 particularly adapted for administering a therapeutic composition which comprises a power input element 310 configured to connect with and receive power from a power source. A power adjustment device 320 is connected to the power input element 310. as well as to an optional data input element 325 connected to a data processing device 330. A power output element 340 is connected to the power adjustment device 320 and is configured to connect with and provide power to a processing element. An optional power-storing device 350 is configured to couple with the power input element 310.

FIG. 4 shows an exemplary controlling device 401 threaded with and/or connected to a vaporizing element or a spraying element 402 for administering a therapeutic composition. The vaporizing element or spraying element 402 comprises a power source 415, an operating button 460, and a heating element 475 configured to heat the package containing therapeutic composition 485. The vaporizing element or spraying element 402 further includes a vaporizing chamber 490 and an aspiration through opening 498. When an operating button 460 is pressed, air is caused to pass through air intake 492, and be mixed with therapeutic composition held in package 485 containing therapeutic composition (which is heated by heating element 475), and to enter the vaporizing chamber 490 or the spraying element via opening 494, and then passes via opening 496 and is aspired via opening 498. The controlling device 401 further contains a power input element 410 configured to connect with and receive power from a power source 415, a power adjustment device 420 connected to the power input element 410, and optionally a data input element 425 connected to a data processing device 430. The controlling device 401 further contains a power output element 440 that is configured to be controlled by the power adjustment device 420 and to provide power to the power connecting element 445. An optional power-storing device 450 is configured to couple with the power input element 410. An optional electric transformer (not shown) is configured to couple with the power source 415. The controlling device 401 is configured to be readily removable from the power source 415 and the processing element. One can attach or connect, e.g., removably connect, the controlling device 401 to the vaporizing element or a spraying element 402. The power source 415 is connected to the heating element 475 which is controlled by to the controlling device 401. This exemplary controlling device 401 can also be fitted to a vaporizing element or a spraying element with detachable power source of the type shown in FIG. 6.

FIG. 5 and FIG. 6 show an exemplary vaporizing element or a spraying element that can be used in with some exemplary embodiments of the present invention. The exemplary vaporizing element or spraying element shown in FIGS. 5 and 6 can optionally (but need not) be used with the invention. FIG. 5 shows an exemplary vaporizing element or a spraying element 500. The vaporizing element or a spraying element 500 comprises a power section 502 that includes a power source 515, an operating button 560, and a heating element 575 which is configured to heat the package containing therapeutic composition 585 (containing oil, herb or wax). Pressing the operating button 560 directs power from the power source 515 to heating element 575, causing the heating element 575 to heat the material inside package 585. The heating of the material inside 585 generates vapors or spray. The vaporizing element or spraying element 500 further includes a vaporizing chamber 590. An aspiration through opening 598 is arranged on one end. When an operating button 560 is pressed, air is caused to pass through air intake 592 and to be mixed with vapors or spray released from package 585 (heated by heating element 575), and to enter the vaporizing chamber 590 or the spraying element via opening 594, and then passes via opening 596 and is aspired via opening 598.

FIG. 6 shows an exemplary vaporizing element or a spraying element 600 that can also be optionally used with the invention. The vaporizing element or a spraying element 600 comprises a power section 602 that includes a power source 615, an operating button 660, and a heating element 675 which is configured to heat the package containing therapeutic composition 685 (containing oil, herb or wax). Pressing the operating button 660 directs power from the power source 615 to heating element 675, causing the heating element 675 to heat the material inside package 685. The heating of the material inside the package 685 generates vapors or spray. The vaporizing element or spraying element 600 further includes a vaporizing chamber 690. An aspiration through opening 698 is arranged on one end. When operating button 660 is pressed, air is caused to pass through air intake 692 and to be mixed with vapors or spray released from package 685 (heated by heating element 675), and to enter the vaporizing chamber 690 or the spraying element via opening 694, and then to pass via opening 696 and is aspired via opening 698.

Commercial vaporizing elements or spraying elements are generally of two types, (i) ones containing an internal power source wherein a power source cannot be detached from the vaporizing element or a spraying element as shown in the example of FIG. 5, and (ii) a detached kind wherein the power source and optionally the heating element can be detached from the material package and the vaporizing chamber as shown in the example of FIG. 6. Non-limiting examples of commercially available vaporizing elements or spraying elements can include: DI vape pen from MIG VAPOR; eGO A10 Vape Pen from Joyetach; Stick V8 Big Baby Vape Pen from SMOK; Evolve-D Dry Herb Vape Pen form Yocan, Eco Fyhit From Herbstick; CE3 Mini 520 Vape pen from BUD; Airis MW Pod from Airistech. The details and specifications for each of these devices are herein expressly incorporated by reference in their entireties. FIG. 5 schematically shows an exemplary vaporizing element or a spraying element with an internal power source. FIG. 6 schematically shows an exemplary vaporizing element or a spraying element with detachable power source.

Thus, the scope of the invention shall include all modifications and variations that may fall within the scope of the attached claims. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed:

1. A controlling device particularly adapted for administering a therapeutic composition, comprising (i) a power input element configured to connect with and receive power from a power source; (ii) a power adjustment device connected to said power input element optionally comprising a data input element connected to a data processing device; (iii) a power output element connected to said power adjustment device and configured to connect with and provide power to a processing element; optionally (iv) a data output element connected to the data processing device and connected to said power output element; optionally (vi) a power-storing device configured to couple with said power input element, and optionally (vii) an electric transformer configured to couple with a power source, wherein said power output element is configured to be controlled by said power adjustment device and wherein said controlling device is configured to be readily removable from the power source and the processing element.

2. A controlling device according to claim 1, wherein said processing element comprises a controllable unit selected from a heating unit, a gas or liquid input opening, an additive input opening and combinations thereof and wherein said controllable unit is configured to receive power from said power output element.

3. An article for administering a therapeutic composition by vaporizing and/or spraying comprising the controlling device according to claim 1, and a processing element, wherein said processing element comprises a chamber and a heating unit set up to heat said chamber and/or the content of said chamber and wherein said chamber comprises an opening designed for input of said therapeutic composition, an opening for output of vapors, or both.

4. An article particularly adapted for administering to a patient a therapeutic composition by vaporizing or by spraying, comprising (i) a vaporizing element or a spraying element configured to vaporize or to spray said therapeutic composition and optionally to activate the therapeutic composition, wherein said vaporizing element or said spraying element optionally comprises a heating unit; (ii) a power input element configured to connect to and receive power from a power source; (ii) a data input element connected to the power input element and to a data processing device; (iii) a power output element connected to said data input element and configured to connect with and provide power to said vaporizing element or to said spraying element; optionally (iv) a data output element connected to the data processing device and connected to said power output element; optionally (vi) a power-storing device configured to couple with said power input element, and optionally (vii) an electric transformer configured to couple with a power source, and, wherein said power output element is configured to be controlled by said data input element.

5. An article according to claim 4, wherein said vaporizing element comprises a vaporizing chamber and a heating unit configured to heat said chamber and/or the content thereof, which heating unit is configured to be powered by said power output.

6. An article according to claim 4, wherein said vaporizing element comprises at least one of a display and a temperature measurement device, and wherein said temperature measurement device is configured to provide data to said data processing device, optionally via said data output element or wherein said spraying element comprises at least one of a display and a flow measurement device, which flow measurement device is configured to provide data to said data processing device, optionally via said data output element.

7. An article according to claim 6, wherein said vaporizing chamber comprises an input opening, which opening is configured to receive said therapeutic composition from a package containing said therapeutic composition or wherein said spraying element comprises an input opening, which opening is configured to receive a sprayable composition from a package containing said therapeutic composition, wherein said package includes processing parameters data, wherein said data processing device is configured to read said processing parameters data and wherein said data processing device is configured to control said power output element according to said processing parameters and according to patient personal data.

8. An article according to claim 4, wherein the data output element is configured to provide data to said data processing device, optionally via said data output element, and wherein said data is selected from the group consisting of processing parameters, vaporizing dates, vaporizing hours, spraying dates, spraying hours, connecting and/or disconnecting data from said power source, connecting and/or disconnecting data from said vaporizing element or said spraying elements and combinations thereof.

9. An article according to claim 4, wherein said data processing device is configured to store data received from said data output element and patient personal data optionally to process said received and stored data and optionally to share said processed data with a central medical records processing device.

10. An article according to claim 9, wherein said vaporizing chamber or said spraying element comprises an input opening configured to receive said therapeutic composition from a package containing said therapeutic composition and wherein said vaporizing chamber or said spraying element further comprises a gas or a liquid input opening, which gas or liquid input opening is configured to introduce a gas a or liquid to said vaporizing unit or said spraying element, and wherein said package containing said therapeutic composition includes processing parameters data, and wherein said data processing device is configured to read said processing parameters data and wherein said data processing device is configured to control said gas or a liquid input opening according to said processing parameters and according to patient personal data.

11. An article according to claim 9, wherein said vaporizing chamber or said spraying element comprises an additive input opening configured to introduce an additive to said vaporizing unit and wherein said additive input opening is configured to be controlled by said data processing unit.

12. A method for the controlled administering of a therapeutic composition to a patient, comprising:
(i) providing said therapeutic composition, optionally in a package including said processing parameters;
(ii) collecting processing parameters input data, optionally from said package, and storing said collected input data on a data processing device;
(iii) introducing said therapeutic composition into a processing element; and
(iv) processing said therapeutic composition in said processing element to form vapors or a spray comprising said therapeutic composition;
wherein said processing is powered by an adjustable power source and wherein said adjustable power source is controlled by said data processing device.

13. A method according to claim 12, wherein said therapeutic composition comprises at least one of a cannabinoid, a solution of a cannabinoid in a solvent, a cannabis plant material, a cannabis extract and a solution of said extract in a solvent.

14. A method according to claim 12, further comprising collecting operating parameters output data, optionally processing said collected output data on a data processing device and optionally sharing said processed data with a central medical records processing device.

15. A method according to claim 12, wherein said processing comprises evaporating and optionally activating in a processing element comprising a vaporizing chamber and a heating unit configured to heat said chamber and/or the content thereof, wherein such heating is powered by said power output.

16. A method according to claim 15, further comprising introducing a gas or a liquid into said vaporizing chamber through a gas or a liquid input opening controlled by said data processing unit.

17. A method according to claim 15, further comprising introducing an additive into said vaporizing chamber through an additive input opening controlled by said data processing unit.

18. A method according to claim 17, wherein said additive comprises a terpene.

19. A method according to claim 12, wherein said processing comprises spraying in a spraying chamber, which spraying is powered by said power output.

20. A method according to claim 19, further comprising introducing an additive into said spraying chamber through an additive input opening controlled by said data processing unit.

21. A method according to claim 20, wherein said additive comprises a terpene.
